(19) United States
(12) Patent Application Publication
(10) Pub. No.: US 2022/0295840 A1
(43) Pub. Date: Sep. 22, 2022

(54) MODULATED VEGETABLE PROTEIN

(71) Applicants: SODIMA, Boulogne-Billancourt (FR); INSTITUT NATIONAL DE LA RECHERCHE AGRONOMIQUE (INRA), Paris (FR); INSTITUT DES SCIENCES ET INDUSTRIES DU VIVANT ET DE L'ENVIRONNEMENT, Paris (FR)

(72) Inventors: Sébastien Fraud, Saint Rambert d'Albon (FR); Cynthia El Youssef, Thiverval Grignon (FR); Pascal Bonnarme, Montigny Le Bretonneux (FR); Sophie Landaud-Liautaud, Villiers Saint Frédéric (FR); Sandra Helinck, Villebon sur Yvette (FR)

(73) Assignees: SODIMA, Boulogne-Billancourt (FR); INSTITUT NATIONAL DE LA RECHERCHE AGRONOMIQUE (INRA), Paris (FR); INSTITUT DES SCIENCES ET INDUSTRIES DU VIVANT ET DE L'ENVIRONNEMENT, Paris (FR)

(21) Appl. No.: 17/631,230

(22) PCT Filed: Jul. 31, 2019
(86) PCT No.: PCT/IB2019/000895

§ 371 Date: Jan. 28, 2022

(51) Int. Cl. A23L 11/50; A23L 11/30; A23L 27/24; A23L 7/10; A23L 33/14; A23L 33/185

(52) U.S. Cl. CPC A23L 11/50; A23L 11/37; A23L 27/24; A23L 7/10; A23L 33/14; A23L 33/185

(57) ABSTRACT

A modulated protein composition is described with improved flavor properties over a vegetable protein. Methods of making a modulated protein composition including the use of a volatile modulating yeast culture to ferment a vegetable protein to produce the modulated protein composition are described. Also disclosed are a fermented vegetable composition made from a modulated protein composition, and ingredients and foods including a fermented vegetable composition or a modulated protein composition.

### BACKGROUND

Vegetable proteins offer an opportunity to be used as a substitute or a supplement for animal or dairy proteins. However, while purification and processing of such vegetable proteins has, in some cases, improved functionality and flavor, vegetable proteins still commonly suffer from poor flavor in various food applications. Although consumers want the ability to consume foods that include plant protein, they generally do not prefer many of the flavors associated with vegetable proteins. Thus, there is a need to improve the flavor profile of vegetable proteins for use in food applications.

### SUMMARY

The present disclosure relates to a modulated vegetable protein with modulated volatile content.

A method of making a modulated protein composition is disclosed herein. The method includes providing a modulation mixture, comprising a vegetable protein and a volatile modulating yeast culture, and fermenting the modulation mixture under volatile modulation conditions to form the modulated protein composition.

In some embodiments, the modulation mixture can further include a lactic acid bacteria culture.

In some embodiments, a method of making a modulated protein composition can further include combining the modulated protein composition with a lactic acid bacteria culture to form a fermentation mixture, and fermenting the fermentation mixture under fermentation conditions to form a fermented vegetable protein.

In some embodiments, the vegetable protein can include legume protein, such as pea protein.

In some embodiments, volatile modulation conditions can include a temperature of 25° C. to 45° C. In some embodiments, volatile modulation conditions can include a period of time of 5 hours to 20 hours.

In some embodiments, a method of making a modulated protein composition can further include inactivating the volatile modulating yeast culture. In some embodiments, inactivating the volatile modulating yeast culture can include heating the modulated protein composition at a temperature and time sufficient to inactivate the volatile modulating yeast culture.

In some embodiments, fermentation conditions can include a temperature of 25° C. to 45° C. In some embodiments, fermentation conditions can include a period of time of 5 hours to 24 hours.

In some embodiments, the volatile modulating yeast culture can modulate off-flavor molecule content, such as aldehyde content, alcohol content, ketone content, or furan content. In some embodiments, the volatile modulating yeast culture can significantly decrease overall ketone content.

In some embodiments, the volatile modulating yeast culture can modulate heptanal content, hexanal content, pentenol, heptanone, or furan content. In some embodiments, the volatile modulating yeast culture can significantly decrease (E)-2-heptanal content, (E)-2-hexanal content, 1-penten-3-ol content, 6-methyl-5-hepten-2-one content, or trans-2-(2-pentenyl)furan content.

In some embodiments, the volatile modulating yeast culture can significantly increase fruity ester content.

In some embodiments, the volatile modulating yeast culture can include a Kluyveromyces species, a Torulaspora species, or a Yarrowia species. In some embodiments, the volatile modulating yeast culture can include Kluyveromyces marxianus, Kluyveromyces lactis, or Torulaspora delbrueckii.

In some embodiments, the modulated protein composition can contain measurable amounts of at least 5 different fruity ester molecules.

In some embodiments of making a modulated protein composition, the method can further include drying the modulated protein composition to produce a powder.

In some embodiments of making a modulated protein composition, the method can further include drying a fermented vegetable protein to produce a powder.

A composition is also disclosed herein. The composition is produced according to a method described herein.

Also described is a composition comprising a vegetable protein including deactivated volatile modulating yeast.

In some embodiments, the vegetable protein can contain measurable amounts of at least 5 different fruity ester molecules.

Also disclosed is a composition comprising a vegetable protein including a volatile modulating yeast.

A food product is disclosed herein. The food product includes a composition described herein. In some embodiments, the food product is a cereal-based food. In some embodiments, the food product is a dairy or non-dairy fermented food.

These and various other features and advantages will be apparent from a reading of the following detailed description.

### DRAWINGS

FIG. 1 shows a comparison of the number of volatile molecules detected by GCMS in an uninoculated modulation mixture, a LAB fermented modulation mixture, and a modulated protein composition (K. marxianus+LAB fermentation).

### DETAILED DESCRIPTION

Many consumers prefer to avoid eating animal-based foods, including those based on milk ingredients and meats. Plant-based protein alternatives to animal-based proteins are available, including proteins from soybeans, almonds, peas, and the like. However, available plant proteins often suffer from poor flavor, and food products made from them, such as non-dairy yogurt, often have poor flavor and/or low protein content.

It was discovered, and is disclosed herein, that a vegetable protein can be fermented with a volatile modulating yeast culture to modulate the volatile content of the vegetable protein to improve flavor. In particular, a modulated protein composition or a fermented vegetable protein described herein has a flavor profile that is significantly reduced in beany notes and/or green notes. In some cases, a modulated protein composition or a fermented vegetable protein provided herein can have a flavor profile that has increased fruity or floral notes. This discovery is particularly surprising because many yeast species are generally considered spoilage organisms in foods, causing off-flavors and off-odors in the foods they contaminate. For example, yeast species suitable for use in the present invention, such as Kluyveromyces species and Torulaspora species, are often considered spoilage organisms in dairy products, such as fresh yogurt, fresh cheese, and cream. Further, although some volatile modulating yeast, such as Kluyveromyces marxianus, are known in the development of kefir grains, their ability to modulate volatile content in proteins, particularly vegetable proteins, was unknown prior to the present invention.

Importantly, vegetable proteins fermented with a volatile modulating yeast culture according to a method provided herein can still retain functionality for use in a food. For example, a pea protein fermented using a volatile modulating yeast culture can be used in a method for making a protein matrix as described in international patent application no. PCT/IB2017/001322. This is surprising since many yeast species have proteolytic activity that can affect the structure and/or function of proteins.

As used herein, the term “volatile modulating yeast culture” refers to a yeast culture that improves a vegetable protein's flavor profile by modulating volatile content of the vegetable protein. A volatile modulating yeast culture is identified by its ability to significantly increase the levels of at least 5 different fruity esters in a modulation test. Fruity esters include any ester that exhibits an aroma or flavor associated with fruit or sweetness. Examples of fruity esters include, but are not limited to: acetic acid, methyl ester (sweet, fruity); isobutyl acetate (fruity, apple, banana); 3-methyl-,acetate-1-butanol (fruit, banana, sweet); 2-methyl-,acetate-1-butanol (fruity, sweet, banana, tropical); hexanoic acid, ethyl ester (pineapple, banana); ethyl formate (sweet, grainy, wine and cognac); acetic acid, ethenyl ester (sweet, fruity); ethyl acetate (fruity); propanoic acid, ethyl ester (fruity); n-propyl acetate (fruity); propanoic acid, 2-methyl-,ethyl ester (sweet, ethereal and fruity); acetic acid, pentyl ester (sweet, fruity, pear, overripe banana); 1-butanol, 3-methyl-,propanoate (sweet, fruity, apple, banana, fresh green tropical); acetic acid, hexyl ester (green, fruity, fatty, sweet, fresh apple, pear); propanoic acid,2-methyl,3-methylbutyl ester (fruity, waxy, apricot, pineapple, green, banana); octanoic acid, ethyl ester (fruity, wine, waxy, sweet, apricot, banana, brandy, pear); acetic acid, 2-phenylethyl ester (floral, rose, honey, sweet, fruity, tropical); propanoic acid, 2-phenylethyl ester (floral, rose, fruity, honey); propanoic acid, 2-methyl-,2-phenylethyl ester (floral, fruity, rose, peach, pastry); and decanoic acid ethyl ester (sweet, waxy, fruity, apple, grape, oily, brandy).

A modulation test includes the following steps:
a. Producing a test composition by combining and mixing a mixture of 4% by weight pea protein isolate (e.g., Purispea TM870 from Cargill) and 3% by weight sucrose in water, and thermally treating the test composition for 15 minutes at 110° C. using an autoclave. The test composition can be refrigerated after thermal treatment and prior to use;
b. Inoculating a sample of the test composition at 30° C. with a yeast culture to be tested (10⁷ CFU per ml) and a lactic acid bacterial (LAB) culture (Danisco® VEGE 047 LYO (Dupont Nutrition & Health, Copenhagen, Denmark) at 20 DCU per 100 L);
c. Inoculating a control sample of the test composition at 30° C. with just the LAB culture (Danisco® VEGE 047 LYO at 20 DCU per 100 L);
d. Incubating the inoculated samples for sufficient time at 30° C. to reach a pH of 4.55;
e. Stopping fermentation of the test samples by placing them in an ice bath for 1 hour, and storing at −80° C. to prevent reactions in the samples prior to performing gas chromatography mass spectrometry (GCMS);
f. Performing gas chromatography mass spectrometry (GCMS) on 5 g of an uninoculated sample of the test composition at 4° C. and 5 g of each of the inoculated samples of the test composition at 4° C. using a non-polar column DB-5MS (60 m×0.32 mm×1 μm) according to the GCMS protocol described in Example 1;
g. Identifying volatile content based on GCMS retention time and mass spectrum as compared to the National Institute of Standards and Technology (NIST) 2017 Mass Spectral Library database; and
h. Comparing fruity ester content of the samples. A volatile modulating yeast culture will have significantly increased levels of at least 5 fruity esters over both the uninoculated test composition sample and the control sample inoculated with only the LAB culture.

Examples of volatile modulating yeast cultures include, for example, Kluyveromyces species cultures (e.g., K. marxianus, K. lactis), Torulaspora species cultures (e.g., T. delbrueckii), Yarrowia species cultures (e.g., Y. lipolityca), Debaryomyces species (e.g., D. hansenii), Candida species (e.g., C. kefir), and Saccharomyces species (e.g., S. cerevisiae). Additional volatile modulating yeast cultures can be identified using a modulation test, as described above. For example, yeast cultures from a collection of yeast (e.g., the Phaff Yeast Culture Collection, University of California, Davis) can be subjected to a modulation test to determine whether any of the yeast cultures are volatile modulating yeast cultures. In some embodiments, yeast cultures can be excluded based on known toxin production or other factors that make them unsuitable for use in producing a food.

A volatile modulating yeast culture can be used herein in a method of making a modulated protein composition. A method of making a modulated protein composition includes providing a modulation mixture. As used herein, a modulation mixture is an aqueous mixture that includes a vegetable protein and a volatile modulating yeast culture.

A vegetable protein can be included in a modulation mixture in an amount sufficient to achieve a protein concentration of from about 2% to about 10% (e.g., about 3% to about 8%, or about 3% to about 6%) by weight of the modulation mixture. A vegetable protein can be included in a modulation mixture in any form, such as a vegetable flour, a protein concentrate, or a protein isolate. Any edible vegetable protein (e.g., protein sourced from: legumes, such as soybean, green pea, yellow pea, lentil, peanut, chickpea, and the like; nuts, such as cashew, almond, and the like; grains, such as wheat, oat, barley, and the like; seeds, such as quinoa, canola, hemp, and the like; and other sources, such as algae, spinach, and the like) or mixtures of vegetable proteins can be used in the invention described herein. However, legume protein, especially pea protein is preferred because such proteins are a readily available source of vegetable protein suitable for many different food applications.

A modulation mixture includes a volatile modulating yeast culture in an amount of 10⁵ (e.g., 10⁶ to 10⁸, or 10⁷) CFU per ml of modulation mixture.

In some embodiments, a modulation mixture can also include a lactic acid bacterial (LAB) culture in an amount of at least 10⁵ (e.g., 10⁶ to 10⁸, or 10⁷) CFU per ml, or at least 10 (e.g., about 10 to 30 Danisco Culture Units (DCU)) per 100 L, of modulation mixture. Any food safe LAB culture can be used that includes one or more lactic acid bacteria species. Examples of useful lactic acid bacteria species include, without limitation, Streptococcus thermophilus, Lactobacillus delbrueckii bulgaricus, Lactobacillus acidophilus, Bifidobacterium animalis lactis, Weissella cibaria, and any combinations thereof. In some embodiments, a combination of Streptococcus thermophilus, Lactobacillus delbrueckii bulgaricus can be included in a modulation mixture.

In some embodiments, a LAB culture can be selected for a desired attribute, such as fermentation rate, preferred fermentation temperature, ability to reach a final pH (e.g., less than about 4.7, or less than about 4.6) contribution to texture (e.g., firmness, viscosity, smoothness, and/or creaminess), contribution to flavor, and/or contribution to appearance of a final product. In some embodiments, a lactic acid bacteria culture can be selected to achieve a desired pH in a time of less than 24 hours (e.g., less than 12 hours, or 8 hours or less, or 6 hours or less).

In some embodiments, a modulation mixture can also include a carbohydrate, such as sugar (e.g., sucrose or lactose) and/or a starch, in an amount of at least 2% (e.g., from about 2% to about 5%) by weight of the modulation mixture. A carbohydrate to be included in a modulation mixture can be selected based on fermentation requirements of a modulating yeast culture and/or LAB culture included in the modulation mixture. In some embodiments, an amount of carbohydrate included in a modulation mixture can be selected based on the amount of carbohydrate needed to achieve a desired pH during fermentation. For example, in some embodiments, a carbohydrate can be included in a modulation mixture in an amount that does not limit fermentation by a volatile modulating yeast culture and/or a lactic acid bacteria culture. In some embodiments, a carbohydrate can be included in an amount that limits fermentation by a volatile modulating yeast culture after a modulation mixture reaches a pH of about 6.

In some embodiments, a modulation mixture can include other ingredients, such as a fat, amino acids, vitamins, and the like. Additional ingredients can be selected based the preferences of the volatile modulating yeast used and/or the lactic acid bacteria culture used.

In some embodiments, ingredients in a modulation mixture can be thermally treated prior to addition of a volatile modulating yeast culture or a LAB culture. Thermal treatment of such ingredients can be for a time and temperature sufficient to inactivate microorganisms in the ingredients. As used herein, the term “inactivation” and its derivatives with reference to a microorganism (e.g., microorganisms in modulation mixture ingredients or volatile modulating yeast culture), refers to rendering the microorganism unable to reproduce, and preferably killing the microorganism. Suitable thermal treatment conditions can be determined using any appropriate methods. Examples of suitable thermal treatment conditions include temperatures of at least 90° C. (e.g., 90° C. to 120° C., 100° C. to 115° C., or about 110° C.) for at least 5 minutes. It is to be understood that thermal treatment can be done for longer periods at lower temperatures to achieve similar results as thermal treatment at higher temperatures and shorter time. In some embodiments, thermal treatment of modulation mixture ingredients can render the ingredients more available to a volatile modulating yeast culture and/or a LAB culture for fermentation under volatile modulation conditions or fermentation conditions as described below.

A modulation mixture is incubated under volatile modulation conditions to form a modulated protein composition. In some embodiments, volatile modulation conditions include a temperature of from about 25° C. to about 45° C. (e.g., about 30° C. to about 43° C.). In some embodiments, volatile modulation conditions can include incubation for a period of time of from about 5 hours to about 20 hours (e.g., about 6 hours to about 12 hours, or about 8 hours to about 10 hours). In some embodiments, volatile modulation conditions can include incubation for a period of time sufficient to achieve a pH of about 6 (e.g., about 5.5 to about 6.5, or about 5.8 to about 6.2) with only a volatile modulating yeast culture (i.e., without a LAB culture). Volatile modulation conditions can be adjusted based on the volatile modulating yeast culture used, whether an LAB culture is included in a modulation mixture, the amount of fermentation time necessary to produce a modulated protein composition, and the like.

As used herein, a modulated protein composition is achieved during fermentation under volatile modulation conditions of a modulation mixture when the modulation mixture has a modulated off-flavor molecule content and/or a significantly increased fruity ester content relative to off-flavor molecule and fruity ester content prior to the start of fermentation. Off-flavor molecules include, for example, aldehydes (e.g., hexanal, (E)-2-hexanal, 2-methylpropanal, octanal, (E)-2-octenal, heptanal, butanal, trans-2-methyl-2-butenal, decanal, (E)-2-heptenal, nonanal, and the like), ketones (e.g., 2,3-octanedione, 6-methyl-5-hepten-2-one, 2-octanone, 2-nonanone, and the like), and furans (e.g., 2-n-heptylfuran, trans-2-(2-pentenyl)furan, 2-ethylfuran, 2-pentylfuran, and the like). In some embodiments, other volatile molecules, such as fruity esters and alcohols (e.g., 1-penten-3-ol, 1-hexanol, 1-octanol, 1-octen-3-ol, (S)-2-heptanol, and the like), can be modulated in a modulated protein composition.

As used herein, the term “modulate” and its derivatives with respect to a content of a molecule or a group of molecules in a modulated protein composition relative to a modulation mixture prior to fermentation, refers to a measurable increase in the content of a molecule or group of molecules, a measurable decrease in the content of a molecule or group of molecules, or a combination of measurable increases and decreases in the content of a molecule or group of molecules. For example, off-flavor molecule content in a modulation mixture can be considered modulated if at least one furan is measurably decreased and an alcohol is measurably increased relative to a modulation mixture prior to fermentation. Increases or decreases in a molecule or group of molecules can be measured using gas chromatography mass spectrometry (GCMS), or other appropriate analytical method.

Without being bound to theory, it is believed that an improved flavor profile of a modulated protein composition is due to both a modulated off-flavor molecule content, as well as an increased ester content, which can result in either reduction of beany and/or green notes, or masking of beany and/or green notes, or both.

While, in some embodiments, LAB culture fermentation can take place during fermentation under volatile modulation conditions if an LAB culture is included in a modulation mixture, it is to be understood that in some embodiments, a modulated protein composition can be further fermented under fermentation conditions using an LAB culture to produce a fermented vegetable protein after fermentation with a volatile modulating yeast culture under volatile modulation conditions. In some embodiments, further fermentation with an LAB culture can be initiated by adding the LAB culture to a modulated protein composition to make a fermentation mixture. In some embodiments, additional ingredients can be included in a fermentation mixture, such as a carbohydrate, additional protein, a fat, or the like.

In some embodiments, fermentation conditions include a temperature of from about 25° C. to about 45° C. (e.g., about 30° C. to about 43° C.). In some embodiments, fermentation conditions can include incubation for a period of time of from about 5 hours to about 24 hours (e.g., about 6 hours to about 18 hours, or about 8 hours to about 12 hours). In some embodiments, fermentation conditions can include incubation for a period of time sufficient to achieve a pH of less than 5 (e.g., about 4.4 to about 4.8, or about 4.5 to about 4.6, or about 4.55). Fermentation conditions can be adjusted based on the LAB culture, desired flavor profile, desired use of the fermented vegetable protein, and the like.

In some embodiments, a volatile modulating yeast culture in a modulated protein composition or a fermented vegetable protein can be inactivated. A volatile modulating yeast culture is considered inactivated if no colonies form when a sample containing the volatile modulating yeast culture is inoculated on a medium preferred by the volatile modulating yeast culture after an appropriate time at an appropriate temperature for growth. For example, a K. lactis culture can be considered inactivated if a sample containing the K. lactis culture is plated on a yeast extract glucose chloramphenicol (YGC) medium agar and incubated at 30° C. for 48 hours.

Inactivation of a volatile modulating yeast culture can be done using any appropriate method, such as thermally treating a modulated protein composition or fermented vegetable protein at a temperature and time sufficient to result in inactivation of the volatile modulating yeast culture. For example, a modulated protein composition or fermented vegetable protein can be heat treated at a temperature of at least 65° C. (e.g., 65° C. for at least 15 minutes, or 70° for 10 minutes). An inactivation method can be determined based on the amount and/or type of volatile modulating yeast culture in the modulated protein composition or fermented vegetable protein.

In some embodiments, a modulated protein composition or a fermented vegetable protein described herein can be dried to form a powder. A dried modulated protein composition or a fermented vegetable protein can have a moisture content of less than 8% (e.g., less than 5%, or less than 3%). Any suitable drying method can be used, including lyophilization, spray drying, and the like. In some embodiments, a dried modulated protein composition can be hydrated and fermented using an LAB culture in a similar manner as described above, and used as-is or dried to form a dried fermented vegetable protein.

A food ingredient comprising a modulated protein composition or a fermented vegetable protein described herein is also disclosed. In some embodiments, a modulated protein composition or a fermented vegetable protein can be used immediately after production or dried prior to use alone as a food, or as one of multiple ingredients in a food. To prolong shelf life and/or reduce microbial activity, a volatile modulating yeast culture in a modulated protein composition or a fermented vegetable protein is preferably inactivated prior to its inclusion in a food. However, in some embodiments, a live volatile yeast modulating culture can be included in a food. In some embodiments, growth of a live volatile modulating yeast culture in a food can be limited by limiting the amount of carbohydrates available to the yeast for fermentation. Available carbohydrate can be limited by limiting the total carbohydrate content, or limiting only the carbohydrates that can be used by the selected volatile modulating yeast culture.

A food ingredient comprising a modulated protein composition or a fermented vegetable protein described herein can be used in any appropriate food. For example, a modulated protein composition can be included in a dairy or non-dairy food, such as a fermented dairy or non-dairy food, or an ice cream, or the like. In another example, a modulated protein composition can be included in a cereal-based food, such as a granola bar, a cake mix, a breakfast cereal, or the like.

A modulated protein composition or a fermented vegetable protein provided herein, or ingredients or foods that include the modulated protein composition or fermented vegetable protein, has a flavor profile that is significantly reduced in beany notes and/or green notes relative to a vegetable protein that is not modulated according to a method provided herein. In some cases, a modulated protein composition or a fermented vegetable protein provided herein, or ingredients or foods that include the modulated protein composition or fermented vegetable protein, can have a flavor profile that has increased fruity or floral notes relative to a vegetable protein that is not modulated according to a method provided herein. Beany, green, fruity, and floral notes in a flavor profile can be detected using a tasting panel. For example, a tasting panel trained using appropriate standard sensory training methods can be used to taste samples of a modulated protein composition or a fermented vegetable protein provided herein, or ingredients or foods that include the modulated protein composition or fermented vegetable protein to determine the presence and relative levels of beany, green, fruity, and floral notes relative to a vegetable protein that is not modulated according to a method provided herein.

### EXAMPLES

### Example 1.

A vegetable protein mixture containing 4% by weight pea protein and 3% sucrose in water was thermally treated at a temperature of 110° C. for 15 minutes to ensure that native flora was inactivated. Modulation mixtures were produced by inoculating thermally treated protein mixture with a volatile modulating yeast culture (Kluyveromyces lactis, Kluyveromyces marxianus, or Torulaspora delbrueckii) in an amount of 10⁷ CFU per ml of the mixture and a LAB culture in an amount of 20 DCU per 100 L of the mixture. The modulation mixtures were incubated at 30° C., 35° C., or 39° C. until pH 4.55 was reached (about 16-19 hours at 30° C., about 9-12 hours at 35° C., and about 7-9 hours at 39° C.) to form a modulated protein composition. Control samples were made by inoculating thermally treated protein mixture with just the LAB culture and incubated under the same conditions as the modulation mixtures until a pH of 4.55 was reached. Samples of each modulated protein composition fermented at 30° C. were subjected to GCMS and compared to an uninoculated sample and a LAB-only control sample fermented at 30° C. GCMS was performed on 5 ml of the uninoculated sample, and 5 g of each of the modulated protein composition and control samples of the test composition.

Briefly, samples were held at −80° C., then allowed to equilibrate at 4° C. for 16 hours, then transferred to a sampling support at 10° C. Volatiles from each sample were extracted using a Gerstel Dynamic Headspace System (DHS) coupled with a Gerstel MultiPurpose Sampler (MPS) Autosampler (Mulheim an der Ruhr, Denmark). The DHS system heated the samples to 40° C. for 3 minutes with agitation at a speed of 500 rpm. The samples were purged with helium flow at 30 mL/min for 10 minutes and analytes (volatile molecules) were collected on sorbent material at 30° C. The sorbent material used for volatile molecule collection was Tenax TA (2, 6-diphenylene oxide polymer) (Gerstel). The sorbent material was dried to remove residual water vapor at 30° C. with a helium flow of 50 mL/minute for 6 minutes. GCMS was performed using a 7890 Agilent GC system coupled to an Agilent 5977B quadruple mass spectrometer (Agilent, Santa Clara, USA). A non-polar Agilent column DB-5MS (60 m×0.32 mm×1μm) was used. Injection was performed in a splitless mode using helium at a flow rate of 1.6 mL/min. The oven temperature of the column was programmed as follows: temperature increase from 40° C. to 155° C. at 4° C./min, then 155° C. to 250° C. at 20° C./min. The oven temperature was then maintained at 250° C. for 5 minutes. The gas chromatogram was recorded and analyzed for volatile retention time.

The chromatogram peak area for off-flavor molecules and fruity esters was recorded. Gas chromatogram peak area for a volatile compound generally correlates with concentration of the volatile compound in the sample in which it is measured. Results for selected off-flavor molecules are shown in Table 1. Results for selected fruity esters are shown in Table 2.

TABLE 1

| Molecule | | Non- | LAB-only | K. lactis + | K. marxianus + | T. delbrueckii + |
|---|---|---|---|---|---|---|
| type | Molecule | fermented | control | LAB | LAB | LAB |
| Aldehyde | Hexanal | 3 × 10⁷ | ND* | ND | ND | ND |
| | (E)-2-hexenal | 4 × 10⁴ | ND | ND | ND | ND |
| | 2- | 2 × 10⁵ | 4 × 10⁵ | 2 × 10⁷ | 2 × 10⁷ | 2 × 10⁷ |
| | methylpropanal | | | | | |
| | Octanal | 8 × 10⁵ | ND | ND | ND | ND |
| | (E)-2-octenal | 3 × 10⁴ | ND | ND | ND | ND |
| | Heptanal | 1 × 10⁶ | ND | ND | ND | ND |
| | Butanal | 2 × 10⁶ | ND | ND | ND | ND |
| | 2-methyl-2- | 4 × 10⁴ | ND | ND | ND | ND |
| | butenal | | | | | |
| | Decanal | 2 × 10⁴ | ND | ND | ND | ND |
| | (E)-2-heptenal | 3 × 10⁴ | 2 × 10⁴ | ND | ND | ND |
| | Nonanal | 3 × 10⁵ | ND | ND | ND | ND |
| Alcohol | 1-penten-3-ol | 2 × 10⁵ | 2 × 10⁵ | ND | 5 × 10⁵ | 3 × 10⁵ |
| | 1-hexanol | 8 × 10⁴ | 7 × 10⁶ | 6 × 10⁶ | 1 × 10⁷ | 7 × 10⁶ |
| | 1-octanol | 6 × 10⁴ | 3 × 10⁵ | 3 × 10⁵ | 4 × 10⁵ | 3 × 10⁵ |
| | 1-octen-3-ol | 2 × 10⁵ | ND | ND | 4 × 10⁵ | 4 × 10⁵ |
| | (S)-2-heptanol | ND | ND | ND | 1 × 10⁶ | 3 × 10⁵ |
| Ketone | 2,3- | 3 × 10⁴ | ND | ND | ND | ND |
| | octanedione | | | | | |
| | 6-methyl-5- | 1 × 10⁵ | 1 × 10⁵ | ND | ND | ND |
| | hepten-2-one | | | | | |
| | 2-octanone | 2 × 10⁵ | ND | 4 × 10⁵ | ND | 4 × 10⁵ |
| | 2-nonanone | 4 × 10⁵ | 5 × 10⁵ | 4 × 10⁵ | ND | 6 × 10⁵ |
| Furan | 2-n-heptylfuran | 3 × 10⁴ | ND | ND | ND | ND |
| | Trans-2-(2- | 3 × 10⁵ | 4 × 10⁵ | ND | 4 × 10⁵ | 4 × 10⁵ |
| | pentenyl)furan | | | | | |
| | 2-ethylfuran | 6 × 10⁶ | 7 × 10⁶ | 7 × 10⁶ | 8 × 10⁶ | 8 × 10⁶ |
| | 2-pentylfuran | 4 × 10⁶ | 1 × 10⁷ | 1 × 10⁷ | 2 × 10⁷ | 1 × 10⁷ |

*ND = not detected

As can be seen in Table 1, K. lactis modulated 5 off-flavor compounds (2-methylpropanal, (E)-2-heptenal, 1-penten-3-ol, 6-methyl-5-hepten-2-one, and Trans-2-(2-pentenyl)furan) relative to both the uninoculated sample and the LAB control. K. marxianus modulated 5 off-flavor compounds (2-methylpropanal, (E)-2-heptenal, (S)-2-heptanol, 6-methyl-5-hepten-2-one, and 2-nonanone) relative to both the uninoculated sample and the LAB control. T. delbrueckii modulated 4 off-flavor compounds (2-methylpropanal, (E)-2-heptenal, (S)-2-heptanol, and 6-methyl-5-hepten-2-one) relative to both the uninoculated sample and the LAB control.

TABLE 2

| | Non- | LAB-only | K. lactis + | K. marxianus + | T. delbrueckii + |
|---|---|---|---|---|---|
| Molecule | fermented | control | LAB | LAB | LAB |
| Ethyl formate | ND** | ND | ND | ND | 1 × 10⁵ |
| Acetic acid, | ND | ND | 6 × 10⁵ | 2 × 10⁵ | 1 × 10⁶ |
| ethenyl ester | | | | | |
| Butyl | ND | ND | 9 × 10⁵ | ND | 8 × 10⁵ |
| isocyanatoacetate | | | | | |
| Ethyl acetate | ND | ND | 5 × 10⁸ | 8 × 10⁸ | 8 × 10⁷ |
| Propanoic acid, | ND | ND | 1 × 10⁷ | 2 × 10⁷ | 8 × 10⁶ |
| ethyl ester | | | | | |
| N-propyl acetate | ND | ND | 3 × 10⁶ | 7 × 10⁶ | ND |
| Heptanoic | ND | ND | ND | ND | 5 × 10⁴ |
| acid, ethyl ester | | | | | |
| Propanoic acid, | ND | ND | 3 × 10⁷ | 4 × 10⁵ | 6 × 10⁴ |
| 2-methyl-, ethyl | | | | | |
| ester | | | | | |
| Isobutyl acetate | ND | ND | 7 × 10⁶ | 2 × 10⁶ | ND |
| 3-methyl-, acetate | ND | ND | 2 × 10⁷ | 1 × 10⁶ | 3 × 10⁵ |
| 1-butanol | | | | | |
| 2-methyl-, acetate | ND | ND | 5 × 10⁶ | 5 × 10⁵ | ND |
| acetic acid | | | | | |
| Acetic acid, | ND | ND | 5 × 10⁵ | ND | ND |
| pentyl ester | | | | | |
| 1-butanol,3- | ND | ND | 9 × 10⁵ | ND | ND |
| methyl-, | | | | | |
| propanoate | | | | | |
| Hexanoic acid, | ND | ND | 1 × 10⁶ | ND | 6 × 10⁵ |
| ethyl ester | | | | | |
| Acetic acid, | ND | ND | 5 × 10⁶ | 1 × 10⁶ | ND |
| hexyl ester | | | | | |
| Propanoic acid, | ND | ND | 4 × 10⁵ | ND | ND |
| 2-methyl, 3- | | | | | |
| methylbutyl ester | | | | | |
| Octanoic acid, | ND | ND | 3 × 10⁵ | ND | ND |
| ethyl ester | | | | | |
| Acetic acid, 2- | ND | ND | 9 × 10⁶ | 3 × 10⁶ | 1 × 10⁵ |
| phenylethyl ester | | | | | |
| Propanoic acid, | ND | ND | 4 × 10⁵ | ND | ND |
| 2-phenylethyl ester | | | | | |
| Propanoic acid, | ND | ND | 4 × 10⁵ | ND | ND |
| 2-methyl, 2- | | | | | |
| phenylethyl ester | | | | | |
| Decanoic acid, | ND | ND | 7 × 10⁴ | ND | ND |
| ethyl ester | | | | | |

**ND = not detected

As can be seen in Table 2, fruity esters were not detectable in either uninoculated or control (LAB only) samples, and each of the tested volatile modulating yeast cultures significantly increased at least 5 fruity esters.

Upon tasting the samples, beany and green flavors were reduced in the samples that were fermented with both a volatile modulating yeast culture and LAB, but LAB alone did not decrease beany and green flavors.

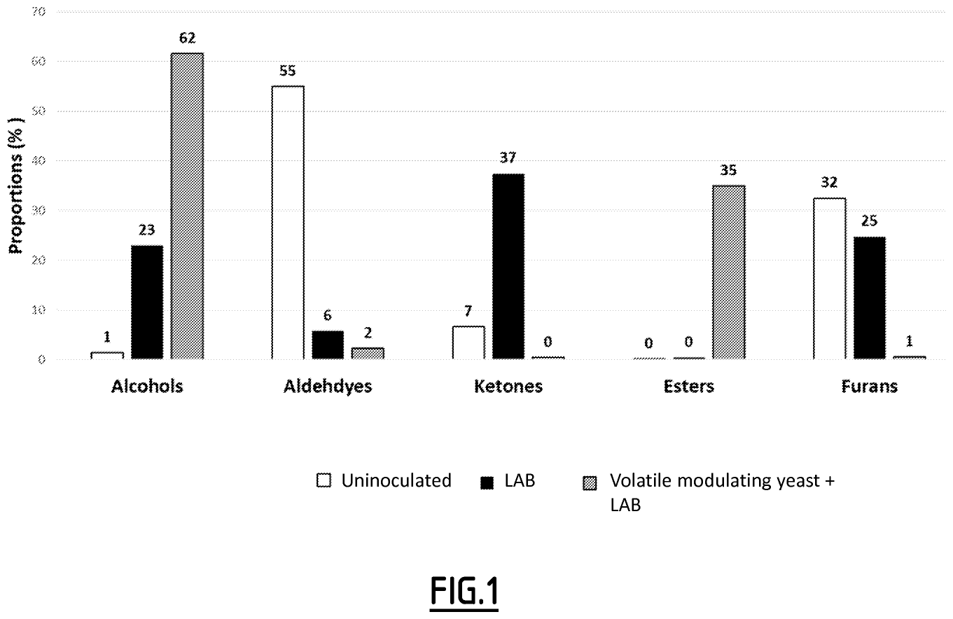

In another experiment, total molecules detection using GCMS was compared between a uninoculated modulation mixture, a LAB fermented modulation mixture, and a modulated protein composition (K. marxianus+LAB fermentation at 30° C. to pH 4.55). FIG. 1 shows the results for the peak chromatogram area of each volatile molecule family (e.g., alcohol family, aldehyde family, ketone family, fruity ester family, and furan family) as a proportion of the peak chromatogram area of all the measured volatiles.

### Example 2

A vegetable protein mixture containing 4% by weight pea protein and 3% sucrose in water was thermally treated at a temperature of 110° C. for 15 minutes to ensure that native flora was inactivated. Modulation mixtures were produced by inoculating thermally treated protein mixture with a volatile modulating yeast culture (Kluyveromyces lactis) in an amount of 10⁷ CFU per ml of the mixture, or both the volatile modulating yeast culture in an amount of of 10⁷ CFU per ml of the mixture and a LAB culture in an amount of 20 DCU per 100 L of the mixture. The modulation mixtures with the volatile modulating yeast culture alone were incubated at 30° C. to a pH of about 6.1 (about 8 hours) and samples were taken for GCMS analysis (labeled “K. lactis, pH 6.1” in Tables 3 and 4), followed by addition of LAB culture in an amount of 20 DCU per 100 L, and further incubated until a pH of about 4.55 was reached, when additional samples were taken for GCMS analysis (labeled “K. lactis, pH pH 6.1/LAB pH 4.55” in Tables 3 and 4). Modulation mixtures containing both volatile modulating yeast culture and LAB culture were incubated at 30° C. until a pH of 4.55 was reached, when samples were taken for GCMS analysis (labeled “K. lactis+LAB pH 4.55” in Tables 3 and 4).

The samples were subjected to GCMS as described in Example 1, and the peak area in the gas chromatogram for off-flavor molecules and fruity esters was recorded. Results for selected off-flavor molecules are shown in Table 3. Results for selected fruity esters are shown in Table 4.

TABLE 3

| | | | | K. | |
|---|---|---|---|---|---|
| | | | | lactis | K. |
| | | | K. | pH 6.1/ | lactis + |
| Molecule | | Non- | lactis, | LAB pH | LAB pH |
| type | Molecule | fermented | pH 6.1 | 4.55 | 4.55 |
| Aldehyde | Hexanal | 3 × 10⁷ | ND⁺ | ND | ND |
| | (E)-2-hexenal | 4 × 10⁴ | ND | ND | ND |
| | 2- | 2 × 10⁵ | 3 × 10⁷ | 4 × 10⁷ | 2 × 10⁷ |
| | methylpropanal | | | | |
| | Octanal | 8 × 10⁵ | ND | ND | ND |
| | (E)-2-octenal | 3 × 10⁴ | ND | ND | ND |
| | Heptanal | 1 × 10⁶ | ND | ND | ND |
| | Butanal | 2 × 10⁶ | ND | ND | ND |
| | 2-methyl-2- | 4 × 10⁴ | ND | ND | ND |
| | butenal | | | | |
| | Decanal | 2 × 10⁴ | ND | ND | ND |
| | (E)-2-heptenal | 3 × 10⁴ | ND | ND | ND |
| | Nonanal | 3 × 10⁵ | ND | ND | ND |
| Alcohol | 1-penten-3-ol | 2 × 10⁵ | 6 × 10⁵ | ND | ND |
| | 1-hexanol | 8 × 10⁴ | 1 × 10⁷ | 1 × 10⁷ | 7 × 10⁶ |
| | 1-octanol | 6 × 10⁴ | 3 × 10⁵ | 4 × 10⁵ | 3 × 10⁵ |
| | 1-octen-3-ol | 2 × 10⁵ | 4 × 10⁵ | 4 × 10⁵ | 4 × 10⁵ |
| | (S)-2-heptanol | ND | ND | ND | 3 × 10⁵ |
| Ketone | 2,3- | 3 × 10⁴ | ND | ND | ND |
| | octanedione | | | | |
| | 6-methyl-5- | 1 × 10⁵ | ND | ND | ND |
| | hepten-2-one | | | | |
| | 2-octanone | 2 × 10⁵ | ND | ND | 4 × 10⁵ |
| | 2-nonanone | 4 × 10⁵ | 1 × 10⁶ | 1 × 10⁶ | 4 × 10⁵ |
| Furan | 2-n-heptylfuran | 3 × 10⁴ | ND | ND | ND |
| | Trans-2-(2- | 3 × 10⁵ | 9 × 10⁵ | 8 × 10⁵ | ND |
| | pentenyl)furan | | | | |
| | 2-ethylfuran | 6 × 10⁶ | 1 × 10⁷ | 1 × 10⁷ | 7 × 10⁶ |
| | 2-pentylfuran | 4 × 10⁶ | 5 × 10⁷ | 3 × 10⁷ | 1 × 10⁷ |

⁺ND = not detected

TABLE 4

| | | | K. | |
|---|---|---|---|---|
| | | | lactis | K. |
| | | K. | pH 6.1/ | lactis + |
| | Non- | lactis, | LAB pH | LAB pH |
| Molecule | fermented | pH 6.1 | 4.55 | 4.55 |
| Acetic | ND⁺⁺ | ND | 7 × 10⁶ | 2 × 10⁶ |
| acid, ethenyl | | | | |
| ester | | | | |
| Ethyl acetate | ND | 4 × 10⁸ | 9 × 10⁸ | 8 × 10⁷ |
| Propanoic | ND | 5 × 10⁶ | 5 × 10⁷ | 2 × 107 |
| acid, ethyl ester | | | | |
| N-propyl acetate | ND | 5 × 10⁶ | 2 × 10⁷ | 7 × 10⁶ |
| Propanoic | ND | 4 × 10⁵ | 4 × 10⁶ | 4 × 10⁵ |
| acid, 2-methyl-, | | | | |
| ethyl ester | | | | |
| Isobutyl acetate | ND | 1 × 10⁶ | 6 × 10⁶ | 2 × 10⁶ |
| 3-methyl-, | ND | 9 × 10⁵ | 6 × 10⁶ | 1 × 10⁶ |
| acetate 1- | | | | |
| butanol | | | | |
| 2-methyl-, | ND | 3 × 10⁵ | 2 × 10⁶ | 5 × 10⁵ |
| acetate 1- | | | | |
| butanol | | | | |
| Acetic acid, hexyl | ND | 2 × 10⁶ | 3 × 10⁶ | 1 × 10⁶ |
| ester | | | | |
| Propanoic | ND | 3 × 10⁶ | 2 × 10⁷ | 3 × 10⁶ |
| acid, 2- | | | | |
| phenylethyl ester | | | | |

⁺⁺ND = not detected

As can be seen in Tables 3 and 4, a volatile modulating yeast culture (in this case, K. lactis) is able to modulate off-flavor molecules and increase fruity ester content on its own.

The implementations described above and other implementations are within the scope of the following claims. One skilled in the art will appreciate that the present disclosure can be practiced with embodiments other than those disclosed. The disclosed embodiments are presented for purposes of illustration and not limitation.

1. A method of making a modulated protein composition, the method including:
a. providing a modulation mixture, comprising a vegetable protein and a volatile modulating yeast culture; and
b. fermenting the modulation mixture under volatile modulation conditions to form the modulated protein composition.

2. The method of claim 1, wherein the modulation mixture further comprises a lactic acid bacteria culture.

3. The method of claim 1, further comprising:
c. combining the modulated protein composition with a lactic acid bacteria culture to form a fermentation mixture; and
d. fermenting the fermentation mixture under fermentation conditions to form a fermented vegetable protein.

4. The method of claim 1, wherein the vegetable protein comprises legume protein.

5. The method of claim 4, wherein the legume protein comprises pea protein.

6. The method of claim 1, wherein the volatile modulation conditions include a temperature in a range of from 25° C. to 45° C.

7. The method of claim 1, wherein the volatile modulation conditions include a period of time in range of from 5 hours to 20 hours.

8. The method of claim 1, further comprising inactivating the volatile modulating yeast culture.

9. The method of claim 8, wherein inactivating the volatile modulating yeast culture comprises heating the modulated protein composition at a temperature and time sufficient to inactivate the volatile modulating yeast culture.

10. The method of claim 3, wherein the fermentation conditions include a temperature in a range of from 25° C. to 45° C.

11. The method of claim 3, wherein the fermentation conditions include a period of time in a range of from 5 hours to 24 hours.

12. The method of claim 1, wherein the volatile modulating yeast culture modulates off-flavor molecule content.

13. The method of claim 12, wherein off flavor molecule content comprises at least one of aldehyde content, alcohol content, ketone content, or furan content.

14. The method of claim 12, wherein the volatile modulating yeast culture significantly decreases overall ketone content.

15. The method of claim 12, wherein the volatile modulating yeast culture modulates at least one of heptanal content, hexanal content, pentenol, heptanone, or furan content.

16. The method of claim 15, wherein the volatile modulating yeast culture significantly decreases at least one of (E)-2-heptanal content, (E)-2-hexanal content, 1 -penten-3-ol content, 6-methyl-5-hepten-2-one content, or trans-2-(2-pentenyl)furan content.

17. The method of claim 1, wherein the volatile modulating yeast culture significantly increases fruity ester content.

18. The method of claim 1, wherein the volatile modulating yeast culture comprises at least one of a Kluyveromyces species, a Torulaspora species, or a Yarrowia species.

19. The method of claim 18, wherein the volatile modulating yeast culture comprises at least one of Kluyveromyces marxianus, Kluyveromyces lactis, or Torulaspora delbrueckii.

20. The method of claim 1, wherein the modulated protein composition contains measurable amounts of at least 5 different fruity ester molecules.

21. The method of claim 1, further comprising drying the modulated protein composition to produce a powder.

22. The method of claim 3, further comprising drying the fermented vegetable protein to produce a powder.

23. A composition produced by the method of claim 1.

24. A composition, comprising a vegetable protein including deactivated volatile modulating yeast.

25. The composition of claim 24, wherein the vegetable protein contains measurable amounts of at least 5 different fruity ester molecules.

26. A composition, comprising a vegetable protein including a volatile modulating yeast.

27. A food product comprising the composition of claim 23.

28. The food product of claim 27, wherein the food product is a cereal-based food.

29. The food product of claim 27, wherein the food product is a dairy or non-dairy fermented food.
